(19) United States
(12) Patent Application Publication
(10) Pub. No.: US 2019/0082953 A1
(43) Pub. Date: Mar. 21, 2019

(54) METHOD OF EVALUATING A RECONSTRUCTED SURFACE, CORNEAL TOPOGRAPHER AND CALIBRATION METHOD FOR A CORNEAL TOPOGRAPHER

(71) Applicant: Stichting VUmc, Amsterdam (NL)

(72) Inventors: Victor Arni de Paz Sicam, Rotterdam (NL); Joris-Joost Snellenburg, Amstelveen (NL); Gerrit Ludolph van der Heijde, Muiderberg (NL)

(21) Appl. No.: 16/127,377

(22) Filed: Sep. 11, 2018

Related U.S. Application Data
(63) Continuation of application No. 12/740,925, filed on Sep. 8, 2010, now abandoned.

(51) Int. Cl. A61B 3/107; G01B 11/25

(52) U.S. Cl. CPC A61B 3/107; G01B 11/25; G01B 11/2513

(57) ABSTRACT

A Method of evaluating a correspondence between a target surface and a reconstructed surface representing the target surface is includes constructing the reconstructed surface by processing information obtained by illuminating the target surface with a pattern of light of a stimulator source, and capturing a reflected image of the pattern of light on an image target. The method further includes the steps of: determining a reference image point on the image target corresponding to a reference stimulator point on the stimulator source; calculating for the reference image point, using the reconstructed surface; a residual representing the correspondence between the target surface and the reconstructed surface; and displaying the residual together with the reflected image.

### CROSS-REFERENCE TO RELATED APPLICATIONS

This application is a continuation of U.S. patent application Ser. No. 12/740,925, filed Sep. 8, 2010, which is the National Stage of International Application No. PCT/EP2007/009666, filed Nov. 2, 2007, the contents of all which are incorporated by reference herein.

### FIELD OF THE INVENTION

Corneal Topography

### BACKGROUND

Measurement of the corneal shape is becoming a common procedure in ophthalmic practice. This is done by a technique called keratoscopy. This technique allows the study of the corneal image and the interpreted image distortions as an indication of an abnormal cornea topography. In keratoscopy the following elements are used: a stimulator source to illuminate a target surface (e.g. the cornea of an eye) with a pattern of light and an image target arranged to receive the reflection of the light pattern. The information from the reflection is used to reconstruct the corneal shape. One of the most commonly used stimulator sources is the Placido ring pattern consisting of a disk with alternating black and white rings. In modern topographers, the reflected image of the target surface is captured by a camera and computer algorithms are applied to process this information to reconstruct the corneal shape. However, this procedure is not without problems. It can e.g. be noted that, when reconstructing the corneal surface, numerical algorithms used in commercially available Placido disk topographers neglect skew ray reflections. This leads to inaccuracy in reconstructing corneal surfaces that are not rotationally symmetric. In Placido disk topography, the corneal shape is reconstructed under the assumption that the reflection occurs in a meridian plane. However, this assumption is valid only if the corneal surface is rotation-symmetric. For non-rotation-symmetric surfaces, skew ray reflections can occur. This means that in Placido-based topography, there is ambiguity in the relationship between the stimulator source points and image points especially when the cornea is not a rotationally-symmetric surface. This ambiguity can e.g. be overcome by applying a stimulator source that enables to establish a one-to-one correspondence between a point on the stimulator source and a point on the image. For Placido-based topographers, this can be implemented by modifying the stimulator pattern to e.g. a checkerboard pattern. It can further be noted that when a colour-coded pattern is used instead of the Placido pattern, a similar correspondence between stimulator source points and image points can be obtained and skew ray ambiguity can be eliminated.

As explained above, topographer that are available today provide for a reconstruction of the target surface (e.g. the corneal surface), using numerical algorithms such as surface fitting using splines or Zernike polynomials. Until now, little attention has been given to developing techniques that allow an easy evaluation of the accuracy of the reconstructed surface. One known technique to evaluate the correctness of surface reconstruction algorithms is described by Halstead et al. in Optom. Vis. Sci. 1995, Vol. 72, pp. 821-827. The reconstructed corneal surface is evaluated by calculating the surface normals of the reconstructed surface and comparing them with the angle bisector between incident and reflected rays for each pair of source point and image point. For the correct surface these two vectors should be identical. If both vectors are not identical, a residual representing the difference between the two vectors can be defined. In the algorithm as described by Halstead, an angle residue (corresponding to the difference between the normal and the angle bisector) is calculated as residual. The residual can further be used to improve the accuracy of the reconstructed surface by e.g. a least-squares fitting.

A drawback of the proposed method is however that it does not provide an easy way to assess the relevance of the calculated difference between the actual (target) surface and the reconstructed surface.

### OBJECT OF THE INVENTION

Therefore, it is an object of the present invention to provide a method for evaluation of a reconstructed surface that enables to assess the relevance of the calculated difference between the actual (target) surface and the reconstructed surface in an easy manner. It is another object of the present invention to provide a corneal topographer that enables the evaluation of a reconstructed surface in an easy manner. It is a further object of the present invention to provide a calibration method for a corneal topographer.

Other objects and advantages of the present invention will become apparent from the description in which embodiments of the present invention are described.

### SUMMARY OF THE INVENTION

According to an aspect of the invention, there is provided a method of evaluating a correspondence between a target surface and a reconstructed surface representing the target surface, the reconstructed surface being constructed by processing information obtained by illuminating the target surface with a pattern of light of a stimulator source, and capturing a reflected image of the pattern of light on an image target, the method comprising the steps of
determining a reference image point on the image target corresponding to a reference stimulator point on the stimulator source,
calculating for the reference image point, using the reconstructed surface, a residual representing the correspondence between the target surface and the reconstructed surface,
displaying the residual together with the reflected image,
evaluating the correspondence between the target surface and the reconstructed surface from the displayed residual.

By applying this method, the accuracy of the reconstructed surface can be assessed more easily because of the visualisation of the residual together with the image. By displaying the residual on the image target, one can also assess which parts of the reconstructed surface suffer from the largest error (or residual) and which parts have a small error. This may be important in case the accuracy requirement of the reconstructed surface is not uniform. As an example, reference can be made to the reconstruction of a corneal surface using a corneal topographer. In such an apparatus, a reconstruction of a corneal surface is determined (e.g. using a numerical algorithm), the reconstructed surface is further to be used in a subsequent surgical procedure to adjust a patient's cornea e.g. by using laser technology. It will be clear to the skilled person that in order for this procedure to be successful, the accuracy of the reconstructed surface is important and should be verified. By visualising the reflected image of the target (e.g. the corneal surface), together with the residuals, the accuracy of the reconstructed surface can be assessed by visual inspection. This visual inspection may also be used to determine any further steps to be taken in e.g. modifying the reconstructed surface to reduce the discrepancy between the actual (target) surface and the reconstructed surface. Note that any method or device suitable can be applied for displaying the residual together with the reflected image. Both can e.g. be displayed on a screen that receives its input from a CCD camera, the camera serving as image target for receiving the reflected image from the target surface. Note that the image target can be any image (or picture) recording or receiving device such as a CCD camera or a video camera. The target surface in general represented the subject that is examined, in case the method is applied for examining a patient's eye, the target surface can e.g. be the cornea of said eye.

The method of evaluating a correspondence between a target surface and a reconstructed surface representing the target surface may equally be described as comprising the steps of
illuminating the target surface with a pattern of light of a stimulator source,
capturing a reflected image of the pattern of light on an image target,
processing information from the previous steps to establish the reconstructed surface,
determining a reference image point on the image target corresponding to a reference stimulator point on the stimulator source,
calculating for the reference image point, using the reconstructed surface, a residual representing the correspondence between the target surface and the reconstructed surface,
displaying the residual together with the reflected image,
evaluating the correspondence between the target surface and the reconstructed surface from the displayed residual.

According to another aspect of the present invention, there is provided a corneal topographer comprising
a stimulator source arranged to, in use, illuminate a target surface with a pattern of light,
an image target arranged to receive the reflected image of the target surface, the corneal topographer further comprising a computational unit arranged to, in use,
construct a reconstructed surface representing the target surface by processing information of the stimulator source and the image received on the image target,
identify a reference image point on the image target corresponding to a reference stimulator point on the stimulator source,
calculate for the reference image point, using the reconstructed surface, a residual representing the correspondence between the target surface and the reconstructed surface,
displaying the residual together with the reflected image.

A corneal topographer according to the present invention differs from conventional topographer in that it enables a.o. a residual calculated from a comparison between an actual surface (e.g. a corneal surface) and a reconstructed surface to be displayed together with the reflected image from the target surface. Both can e.g. be displayed together on a screen of the topographer. As described above, such visual inspection being readily available provides an easy way to assess the accuracy of the reconstruction, it may also be a useful tool in deciding whether or not an adjustment of the reconstructed surface is required or not.

According to yet another aspect of the invention, there is provided a calibration method for a corneal topographer, comprising the steps of
identifying a reference image point on an image generated by reflecting a stimulator source on a reference surface towards an image target corresponding to a reference stimulator point,
calculating the coordinates of the stimulator origin point of the reference image point by backward ray tracing from the image point towards the reference surface and from the reference surface towards the stimulator source,
establishing the geometric relationship between the reference image point and the stimulator origin point.

In order for a corneal topographer to provide an accurate reconstructed surface of e.g. a corneal surface, a corneal topographer needs to be calibrated; the relative position between the different components of the topographer need to be know. As an example, surface reconstruction algorithms may depend on the position of the stimulator source relative to the image target being known. In case this relative position is not known or not sufficiently accurate, the calibration method according to the invention can be applied. As such, inaccuracies or errors occurring during an initial calibration by the manufacturer can be solved. The reference surface for use with the calibration method can e.g. be a substantially spherical surface having a known geometry. The reference surface may also be a corneal surface of an eye of which the geometrical properties are known.

### BRIEF DESCRIPTION OF THE FIGURES

FIG. 1 schematically depicts the basic principle of corneal topography;

FIG. 2 schematically depicts a corneal topographer enabling a one-to-one correspondence between a stimulator point and an image point;

FIG. 3 schematically depicts the principle of backward ray tracing as applied in an embodiment of the present invention;

FIG. 4 schematically depicts the principle of pseudo-forward ray tracing as applied in an embodiment of the present invention.

### DETAILED DESCRIPTION

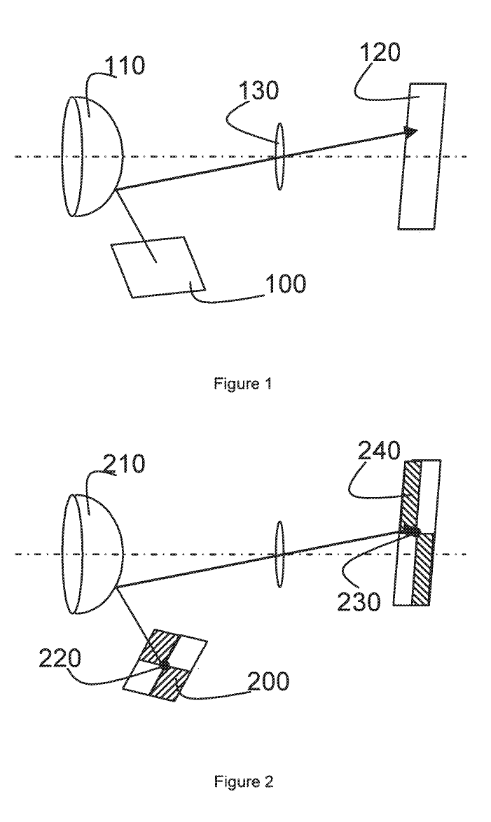

FIG. 1 schematically depicts a part of a stimulator source 100 arranged to illuminate a target surface 110 (the target can e.g. be an eye). The ray of light emanating from the stimulator source is reflected on the target surface, the reflected ray of light is received by the image target 120 which can e.g. be a CCD camera. Between the target surface and the image target, a lens 130 can be present.

In general, when the stimulator source does not comprise distinct feature points, it is not possible to establish a one-to-one relationship between a point on the stimulator source and the corresponding projection on the image. FIG. 2 schematically depicts an arrangement that enables this correspondence. FIG. 2 schematically depicts a part of the stimulator source 200 arranged to illuminate a target surface 210. The part of the stimulator source 200 as shows comprises a pattern of rectangular shapes. The different parts or shapes of the stimulator source can e.g. emit a different colour. Alternatively, the pattern can be an alternating pattern of light and dark shapes. By doing so, a one-to-one correspondence between the stimulator source and the image can be obtained for the so-called crossings (in the stimulator source as depicted, the crossing corresponds to the point where the four rectangles meet) in the stimulator source. In FIG. 2, the crossing (also referred to as stimulator crossing SC) is indicated by reference number 220. As the pattern of the stimulator source is reflected on the target surface and projected on the image target, it will be clear to the skilled person that an image point 230 corresponding to the stimulator crossing can be found on the image 240. This image point is also referred to as the detected crossing DC.

Whether or not such a one-to-one correspondence between a number of stimulator source points and corresponding image points can be established depends on whether or not the stimulator source pattern comprises stimulator crossings that can result in identifiable crossings on the image targets. Topographers that enables such a one-to-one correspondence are e.g. topographers that project a checkerboard pattern, or a dartboard pattern or a colour-coded pattern.

Based on the geometric properties of the stimulator source, i.e. the position of the source relative to the surface target, and the image on the image target, a reconstruction of the target surface can be established. Different ways of achieving such a reconstructed surface exist, such as a curve fitting using Zernike polynomials or a fitting using spline functions.

Once such a reconstructed surface is established (e.g. as a continuous function describing the height of the cornea, or the height deviation from a spherical surface), this can be applied by a surgeon to adjust the shape of a patient's cornea, e.g. using laser refractive surgery procedures. It will be clear that in such a procedure, the outcome may strongly depend on the correspondence between the actual target surface (i.e. the cornea of an eye) and the reconstructed surface.

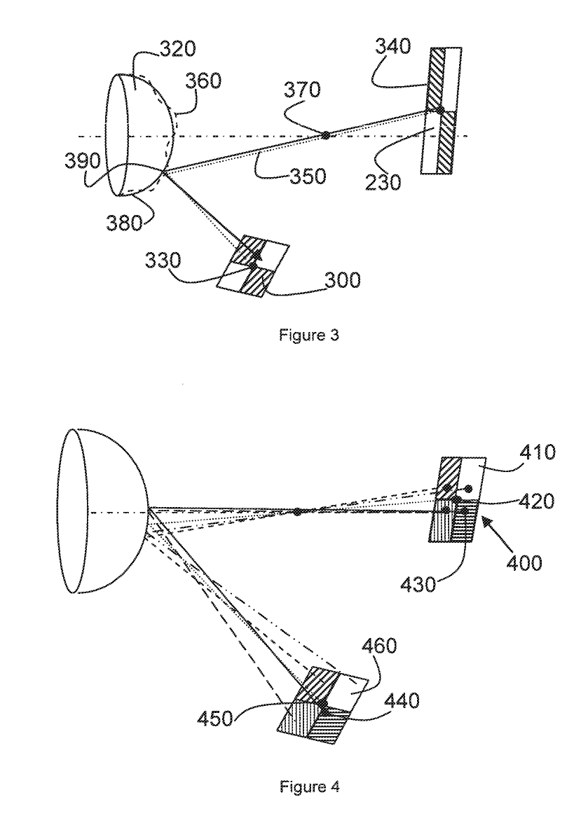

In case the topographer enables that such a one-to-one correspondence for a number of feature point (or crossings) is established, this can be applied to verify the accuracy of a reconstructed surface. One way to check the accuracy of the reconstructed surface is to use a back-ward trace algorithm to trace back the origin of an image point to the stimulator source using the reconstructed surface. This procedure is illustrated in FIG. 3.

FIG. 3 schematically a stimulator source 300 and an image target 310 of a corneal topographer. Also indicated in FIG. 3 are a stimulator crossing 330 and its corresponding detected crossing 340 (i.e. the location on the image target where a ray of light (indicated by the dotted line 350) emanating from the stimulator crossing 320 and reflected on the target surface would end up) on the image target 310. Note that the actual shape of the target surface 320 is indicated by the broken line 360. When the detected crossing 330 is established, it can be traced to the corresponding point (SC) on the stimulator via backward ray-tracing. In this procedure, a ray is traced back from a point on the image through the nodal point of the lens 370 to the reconstructed target surface (indicated by the solid line 380 of the target surface 320. The intersection point 390 on the reconstructed surface can, in general, be calculated because the surface is represented by an analytical function, e.g. as a combination of Zernike polynomials. The correctness of the reconstructed surface can be evaluated by comparing the bisector of the incident ray and reflecting ray with the normal vector on the intersection point. A difference between the two vectors (also referred to as the angle residue) is a measure for the error between the actual surface and the reconstructed surface. In general, any difference between the actual trajectory of a ray of light emanating from the stimulator source and its backward traced ray via the reconstructed surface, can be used as a measure indicating the accuracy of the reconstructed surface. Such a difference is referred to as a residual.

According to an embodiment of the present invention, the residual and the reflected image are displayed together. This provides an easy way for e.g. a surgeon to assess the accuracy of the reconstructed surface and if required, take appropriate measures to improve the reconstructed surface.

According to an embodiment of the present invention the residual comprises an angle residual calculated by backward tracing the reference image point towards the reconstructed surface and from the reconstructed surface towards the stimulator source. The angle residual can e.g. be calculated as indicated above.

According to another embodiment of the present invention, an alternative method to verify the correctness of the surface reconstruction procedure is employed. The procedure is referred to as a pseudo-forward ray tracing (PFRT) routine. The procedure as described in FIG. 3 is referred to as a back-ward trace algorithm because an image point (the detected crossing) is traced back and compared with its point of origin. A forward ray tracing algorithm would trace the stimulator crossing to the image, however, as there are infinitely many rays emanating from the stimulator crossing, forward ray-tracing from this point to the corneal surface would be impossible.

To overcome this, the alternative method according to an embodiment of the present invention applies multiple backward ray-tracing procedures. According to an embodiment of the method, a region around each DC on the image target is considered (in case the image target is a CCD camera plane, the selected region can e.g. be a square region of a predefined number of pixels, e.g. 11×11 pixels). In such an arrangement, each pixel in this region is traced back to the stimulator source using a backward trace algorithm (as e.g. shown in FIG. 3). The pixel with the closest projection to the SC is considered the pixel location of the so-called residual crossing RC. The distance between the residual crossing RC and the detected crossing DC on the image plane can be considered a measure of the accuracy of the reconstructed surface (i.e., this distance can be considered a residual). A visualization of this procedure is shown in FIG. 4. In this figure, 5 pixel points (400) on the image plane 410 are shown: the DC (420) and 4 pixel points in the neighbourhood of the DC. When traced back, pixel 430 is found to have the closest projection (440) to the SC 450 on the stimulator source 460. Thus, pixel 430 is considered as the residual crossing RC. Note that, for clarity, the distance between the pixels, nor the distance to the target surface or stimulator source is not up to scale with the target surface. Note also that the reconstructed surface that is used for backward tracing the different pixels is not separately shown in FIG. 4.

As will be apparent, the proposed method equally provides the possibility of displaying residual information about the accuracy of the reconstructed surface together with the reflected image. Such an on-screen evaluation of the residual (or residual information) together with the image enables an easy assessment of reconstructed surface. As the residual information can be shown together with the reflected image of e.g. the patients eye, one can assess whether or not the reconstructed image is sufficiently accurate, based on the size of the error (measured in e.g. a number of pixels) and the location of the error.

Regarding the latter aspect, it will be clear to the skilled person that a certain error of the reconstructed surface can be acceptable on certain locations of the cornea whereas the same error is unacceptable on other locations. The proposed evaluation method therefore provides the possibility of easily assessing in which areas the reconstructed surface needs further improvement.

A further advantage of the proposed method is that the amplitude of the error (e.g. the distance in pixels between the residual crossing RC and the detected crossing DC is found to be proportional to the corneal height accuracy. Depending on a.o. the pixel size, one can establish the relationship between the error at a certain location on the image plane (expressed in pixels) and the height accuracy (i.e. the distance between the actual (corneal) surface and the recomputed surface in a direction perpendicular to the surface) at that location. As an example, an error of e.g. one pixel may correspond to a height error of 1 micron. Based on this relationship, the proposed alternative method provides a further possibility to easily assess whether a certain error is acceptable or not.

According to yet a further embodiment of the present invention, the residual information obtained by the PFRT algorithm (as indicated in FIG. 4) or the backward trace algorithm (as explained in FIG. 3) is used to further improve the accuracy of the reconstructed surface. As such, the method of evaluating the correspondence between a target surface and a reconstructed surface representing the target surface may comprise the step of modifying the reconstructed surface based on calculated residual.

As an example, it is assumed that the target surface is reconstructed using the well-known Zernike polynomials. A combination of these polynomials (i.e. a summation of the different polynomials weighted by multiplying each polynomial with a corresponding Zernike coefficient) can be used to represent the target surface (e.g. the height of a cornea) as an analytical function. The required coefficients for the weighing of the Zernike polynomials can e.g. be obtained from a least-squares fitting routine. Because initially the surface is unknown, the initial Zernike coefficients can be set equal to zero, describing a flat surface. For each detected crossing on the image, an angle residue can be calculated as the difference between a normal vector (at the intersection point) and an angle bisector between incident and reflected ray, see FIG. 3. The calculated angle residues (or residuals) can then be used in a least-squares fitting procedure to determine a better model for the surface.

As will be clear to the skilled person, the residual of the detected crossings as obtained from the PRFT algorithm can equally be applied in a least-squares fitting routine for obtaining an improved surface. Compared to the use of the angle residues in an optimisation (or further improvement) routine, the use of the residuals of the PRFT algorithm provides the following advantages:
as there is a known relationship between the residual (e.g. expressed in a number of pixels or pixel size) and the height accuracy (as explained above), the calculated residuals can be used to determine whether the optimisation process can be stopped or whether a further iteration is required. In case the residual values are below a certain value, one can easily determine the corresponding height accuracy. Alternatively, one can require a certain height accuracy (e.g. a corneal height error less than 1 micron) and determine the corresponding allowable residual value. This value can be used in the surface optimisation procedure as a criterion to stop or continue the procedure.
As the PRFT algorithm also provides information as to where the errors occur on the corneal surface, a weight function can be applied to the residuals such that an improvement in a next iteration focuses on the areas where an error is least acceptable.

It can further be noted that such a weight function may also be applied to suppress the contribution of large residuals (mainly outliers) in the fitting procedure.

The PFRT method as described above has been applied to measurements of five different surfaces:

(1) a PMMA (polymethyl methacrylate) spherical surface with 6.99 mm radius of curvature.
(2) a PMMA spherical surface with 9.00 mm radius of curvature.
(3) a PMMA toric surface with maximum axial radius of curvature of 8.02 mm and minimum axial radius of curvature of 7.05 mm.
(4) a human cornea, from the left eye of a 38-year-old man, with no known abnormality, and
(5) a human cornea, from the left eye of a 61-year-old man, with subepithelial infiltrate.

As described, the PFRT method can produce residual information in pixel units of the reconstructed surface on the image (e.g. a CCD image) itself. To produce an accurate description of the corneal surface, two things must happen. First, the location of the image crossings (detected crossings DC) must be determined accurately. Second, the numerical reconstruction of the corneal surface must be consistent with the DCs. The output of the PFRT routine is an indicator whether the second procedure was implemented well. It will be clear to the skilled person that the accuracy of the first procedure (obtaining the position of the detected crossings DC) is important to obtain a reliable output of the PFRT procedure or any other evaluation method. In this respect, reference can be made to Spoelder H J W, Vos F M, Petriu E M, Groen F C A. Some aspects of pseudo random binary array-based surface characterization. IEEE Trans. Instrum. Meas. 2000; 49:1331-6 showing that a subpixel accuracy in detecting the location of image crossings DC can be obtained. In case a 1 pixel would correspond to a height accuracy of 1 micron, the PFRT procedure can result in a submicrometer corneal height accuracy when the optimisation is continued until the calculated residuals are less than 1 pixel. In this respect, it is worth mentioning that the accuracy that can be obtained also depends on the complexity of the actual surface combined with the degrees of freedom of the surface fitting function. It has been shown that the overall residual (or mean residual of the detected crossings) increases with complexity of the measured surface. The residuals were found to be the smallest for the artificial surfaces; the spherical surfaces (1) and (2) resulted in a mean residual of 0.70 pixel, the toric surface (3) resulted in a mean residual of 0.81 pixel. The regular cornea (4) was found to have a slightly higher residual compared with the artificial surfaces (a mean residual of 1.16 pixel). This effect is found to be caused by the effect of higher order shape features. However, because these shape features are not as dominant when compared with the spherical and toric shape features, the effect on the residual was found to be relatively small. Whereas, for the irregular cornea (5), the effect of the higher-order shape features is larger, thus producing an increase in the mean value of the residual (a mean residual of 2.94 pixel was found when the surface was modelled using Zernike polynomials until radial order 6). The accuracy of the surface reconstruction was found to improve when the Zernike radial order used to model the corneal surface is increased. The addition of more Zernike components enables better fitting of the local surface features. For the artificial surfaces, a lower radial order for the Zernike expansion (order 6) is sufficient to reconstruct the surface with subpixel accuracy. For the regular cornea, subpixel accuracy was observed only for Zernike radial order of 10 or higher. For the irregular cornea (5), order 20 was found still not sufficient to produce subpixel accuracy for the surface reconstruction. Nevertheless, at this order the accuracy was found to approach pixel resolution, which is reasonable enough for clinical practice. The above also indicates that to some extent the use of Zernike polynomials will produce accurate corneal surface reconstruction as long as a sufficient radial order is used.

It should be noted that the PRFT routine as described does not depend on the way the reconstruction of the surface is done. It will be clear to the skilled person that any surface fitting procedure can be applied to provide a reconstructed surface. This reconstructed surface can then be evaluated using the PFRT routine as described and/or can be further optimised using the outcome of the PFRT routine (i.e. the residual information).

According to an embodiment of the present invention, there is provided in a corneal topographer that enables an evaluation of a reconstructed surface as described above. In order to do so, the corneal topographer comprises a computational unit arranged to
construct a reconstructed surface representing the target surface by processing information of the stimulator source of the topographer and the image received on the image target of the topographer,
identify a reference image point on the image target corresponding to a reference stimulator point on the stimulator source,
calculate for the reference image point, using the reconstructed surface, a residual representing the correspondence between the target surface and the reconstructed surface,
display the residual together with the reflected image.

As will be clear from the above, various way of determining the reconstructed surface or the residual can be applied in such a computational unit. It can further be noted that, in order to identify a reference image point on the image target of the topographer corresponding to a reference stimulator point on the stimulator source of the topographer, various types of stimulator sources can be applied. Examples of such stimulator sources are sources who provide a checkerboard (or dartboard) pattern of light or a colour coded pattern of light. Such a pattern can be obtained by applying different hue-values for the different areas of the pattern, or a different brightness or intensity. A colour coded pattern can be compared to a checkerboard that used areas of different colours as alternative to or in addition to areas that are black or white (i.e. dark and light). The various areas of different colours can be arranged in such manner that a reference image point on the image target of the topographer corresponding to a reference stimulator point on the stimulator source of the topographer can be found more easily.

According to an embodiment of the present invention, a calibration method for a corneal topographer is provided. As explained above, an accurate construction of the reconstructed surface relies on accurate knowledge of the relative position of stimulator source and image target. In order to obtain this knowledge, the topographer can be used with a reference surface as target surface, rather than an unknown surface. Assuming the reference surfaces geometry is known, a corresponding reconstructed surface is also known. Applying any of the tracing routines as explained above on such a surface, should result in a residual substantially equal to zero. If a non-zero residual is found, this means that the initial assumption regarding the geometric relationship between the stimulator source and the image target was incorrect. As the reconstructed surface corresponds provides an accurate representation of the reference surface, backward ray tracing the reference image point towards the stimulator source (via the reconstructed surface) enables the actual co-ordinates of the reference stimulator point (relative to the image target) to be determined. As such, the actual position of the reference stimulator point relative to the corresponding reference image point can be established. It will be clear to the skilled person that in order to obtain the relative position between the stimulator source and the image target in all 6 degrees of freedom, the calibration can be performed for a plurality of reference stimulator points.

Although the examples that are described relate in particular to corneal topography, it can be stated that the methods as described (either the calibration method or the method for evaluating a reconstructed surface) may also be applied in other field of technology where accurate knowledge of the shape of a target surface is required. An example of such a field being semiconductor technology wherein an accurate knowledge of the surface characteristics of a substrate (such as a wafer) is required. Another field where the described methods may be applied is biometrical identification using e.g. an iris scan.

It can further be stated that the method of evaluating the correspondence between a target surface and a reconstructed surface representing the target surface may advantageously be combined with the calibration method as described. Since the calibration method enables an accurate determination of the geometrical relationship between the various components of the topographer, it may be advantageous to apply this calibration prior the reconstructed surface evaluation method as the geometric relationship between the stimulator source and the image target is used in determining the reconstructed surface.

1.-20. (canceled)

21. A method of evaluating a difference between a target corneal surface and a reconstructed corneal surface representing the target corneal surface, the method comprising the steps of:
providing a stimulator source comprising a source pattern, the source pattern comprising a plurality of stimulator points;
illuminating the target corneal surface with a pattern of light from the stimulator source, the pattern of light corresponding to the source pattern of the stimulator source, thereby providing a plurality of target points corresponding to the respective plurality of stimulator points;
capturing a reflected image of the pattern of light, reflected off the target corneal surface, on an image target of an imaging device to form a detected image, the detected image comprising a plurality of detected image points corresponding to the respective plurality of target points;
identifying a one-to-one correspondence between the detected image points on the image target of the imaging device and the stimulator points on the stimulator source;
processing the detected image points to construct the reconstructed corneal surface;
identifying a plurality of test points on the image target in proximity to each detected image point;
backward ray-tracing the plurality of test points from the image target towards the reconstructed corneal surface and from the reconstructed corneal surface towards the stimulator source;
selecting for each detected image point the test point which traces closest to its corresponding stimulator point;
defining the residual based upon the difference between the detected image point and the selected test point origin; and
displaying the plurality of residuals together with the reflected image of the target corneal surface.

22. The method according to claim 21, wherein the stimulator source comprises a colour-coded pattern to form the stimulator points.

23. The method according to claim 21, wherein the source pattern comprises a plurality of features having boundaries disposed about crossing points, the stimulator points being formed by the crossing points.

24. The method according to claim 21, wherein the residual comprises an angle residual calculated by backward ray-tracing a plurality of detected image points from the image target towards the reconstructed corneal surface, and from the reconstructed corneal surface towards the stimulator source, to a plurality of corresponding stimulator points, the residual being the difference in angle between the angle bisector of the ray-traced incident ray and reflected ray and the normal vector of the reconstructed surface on the intersection point of the ray with the reconstructed surface.

25. The method according to claim 21, wherein the residual comprises an stimulator residual calculated by backward ray-tracing a plurality of detected image points from the image target towards the reconstructed corneal surface, reflected off the reconstructed corneal surface towards the stimulator source, calculating a plurality of stimulator test points being the intersection of the reflected ray and the stimulator source, the residual being the difference in distance between the plurality of calculated stimulator test points and the stimulator source points corresponding to the plurality of detected image points.

26. The method according to claim 21, wherein the construction of the reconstructed corneal surface comprises a surface fitting using Zernike Polynomials.

27. The method according to claim 21, wherein the construction of the reconstructed corneal surface comprises a surface fitting using spline functions.

28. The method according to claim 21, further comprising the step of modifying the reconstructed corneal surface based on calculated residual.

29. The method according to claim 28, wherein the step of modifying the reconstructed corneal surface comprises a least-squares fitting.

30. The method according to claim 21, wherein the method is preceded by a calibration method comprising the steps of:
identifying a reference image point on an image target generated by reflecting a stimulator source pattern on a reference surface towards the image target, the reference image point corresponding to a reference stimulator point,
calculating the coordinates of the reference stimulator point of the reference image point by backward ray tracing from the reference image point towards the reference surface and from the reference surface towards the stimulator source, and
establishing the geometric relationship between the reference image point and the reference stimulator point.

31. The method according to claim 21, further comprising a determination of a mathematical relationship between a corneal height accuracy and residual error at a certain location.

32. The method according to claim 21, further comprising:
providing a lens;
wherein when capturing the reflected image of the pattern of light, the pattern of light travels through the lens present between the target corneal surface and the image target, the lens not part of the target surface and not part of the image target.

33. A corneal topographer system, comprising:
a stimulator source comprising a source pattern, the source pattern comprising a plurality of stimulator points;
an illuminator for illuminating a target corneal surface with a pattern of light from the stimulator source, the pattern of light corresponding to the source pattern of the stimulator source, thereby providing a plurality of target points corresponding to the respective plurality of stimulator points;
an imaging device having an image target for capturing a reflected image of the pattern of light, reflected off the target corneal surface, to form a detected image, the detected image comprising a plurality of detected image points corresponding to the respective plurality of target points; and
a computational unit configured for use with the method of claim 21.

34. A corneal topographer system, comprising:
a stimulator source comprising a source pattern, the source pattern comprising a plurality of stimulator points;
an illuminator for illuminating a target corneal surface with a pattern of light from the stimulator source, the pattern of light corresponding to the source pattern of the stimulator source, thereby providing a plurality of target points corresponding to the respective plurality of stimulator points;
an imaging device having an image target for capturing a reflected image of the pattern of light, reflected off the target corneal surface, to form a detected image, the detected image comprising a plurality of detected image points corresponding to the respective plurality of target points; and
a computational unit configured, in use, for:
identifying a one-to-one correspondence between the detected image points on the image target of the imaging device and the stimulator points on the stimulator source;
processing the detected image points to construct the reconstructed corneal surface;
identifying a plurality of test points on the image target in proximity to each detected image point;
backward ray-tracing the plurality of test points from the image target towards the reconstructed corneal surface and from the reconstructed corneal surface towards the stimulator source;
selecting for each detected image point the test point which traces closest to its corresponding stimulator point;
defining the residual based upon the difference between the detected image point and the selected test point origin; and
displaying the plurality of residuals together with the reflected image of the target corneal surface.

35. A method of evaluating a difference between a target corneal surface and a reconstructed corneal surface representing the target corneal surface, the method comprising the steps of:
(a) providing a stimulator source comprising a source pattern, the source pattern comprising a plurality of stimulator points;
(b) performing a calibration method comprising the steps of:
(i) identifying a reference image point on an image target generated by reflecting the stimulator source pattern on a reference surface towards the image target, the reference image point corresponding to a reference stimulator point,
(ii) calculating the coordinates of the reference stimulator point of the reference image point by backward ray tracing from the reference image point towards the reference surface and from the reference surface towards the stimulator source, and
(iii) establishing the geometric relationship between the reference image point and the reference stimulator point;
(c) illuminating the target corneal surface with a pattern of light from the stimulator source, the pattern of light corresponding to the source pattern of the stimulator source, thereby providing a plurality of target points corresponding to the respective plurality of stimulator points;
(d) capturing a reflected image of the pattern of light, reflected off the target corneal surface, on an image target of an imaging device to form a detected image, the detected image comprising a plurality of detected image points corresponding to the respective plurality of target points;
(e) identifying a one-to-one correspondence between the detected image points on the image target of the imaging device and the stimulator points on the stimulator source;
(f) processing the detected image points to construct the reconstructed corneal surface;
(g) identifying a plurality of test points on the image target in proximity to each detected image point;
(h) backward ray-tracing the plurality of test points from the image target towards the reconstructed corneal surface and from the reconstructed corneal surface towards the stimulator source;
(i) selecting for each detected image point the test point which traces closest to its corresponding stimulator point;
(j) defining the residual based upon the difference between the detected image point and the selected test point origin; and
(k) displaying the plurality of residuals together with the reflected image of the target corneal surface.
